(19) United States
(12) Patent Application Publication
(10) Pub. No.: US 2018/0296357 A1
(43) Pub. Date: Oct. 18, 2018

(54) AN EXPANDABLE CAGE

(71) Applicant: TOBB EKONOMI VE TEKNOLOJI UNIVERSITESI, Ankara (TR)

(72) Inventors: Mesut Emre YAMAN, Ankara (TR); Teyfik DEMIR, Ankara (TR); Tolga TOLUNAY, Ankara (TR)

(21) Appl. No.: 15/770,358

(22) PCT Filed: Oct. 24, 2016
(86) PCT No.: PCT/TR2016/000150

§ 371 Date: Apr. 23, 2018

(30) Foreign Application Priority Data
Oct. 26, 2015 (TR) 2015/133645

(51) Int. Cl. A61F 2/44

(52) U.S. Cl. CPC A61F 2/441; A61B 17/7058; A61F 2/4455; A61F 2/4425

(57) ABSTRACT

An expandable cage is placed between two vertebrae and expandable at a preferred ratio towards the vertebrae (i.e. in longitudinal axis). The expandable cage has at least one protrusion which is provided in the body and which, in case of a force being applied, forms a pointed tip out of the body, and at least one balloon which applies the required force to the protrusion such that the protrusion will move unilaterally.

### TECHNICAL FIELD

The present invention relates to an expandable cage which is placed between two vertebrae and capable of expanding towards the vertebrae (in longitudinal axis) at the preferred ratio; and also moves in one direction, i.e. unilaterally, by means of a balloon.

### PRIOR ART

Discs made of articular fibrocartilage and allowing the vertebrae to move easily with respect to one another are present between human vertebrae. Two adjacent vertebrae can easily move relative to one another thanks to said discs, in six degrees of freedom. Discs are present between all successive vertebrae, thereby allowing a limited movement of the vertebral column. In case damage occurs in the vertebrae or vertebral column due to an accident, a tumor incidence, or old age, a part of the disc may be dislocated and apply pressure on the nerve roots and/or the spinal cord. In such cases, the so-called discectomy procedure is performed in which the whole, or a part of the disc is removed surgically. There remains a space between the vertebrae subsequent to discectomy. Said space results in a decrease in the paravertebral disc height. In this case, it may be desirable to join the related vertebral segments together by means of fusion. When fusion is to be performed, an interbody fusion device is positioned in said space such that it will maintain paravertebral height after discectomy. These devices are also referred to as vertebral cages.

Today, many of the cages used for filling the space between two vertebrae are not capable of expanding. In case of placing a cage that is not capable of expanding between the vertebrae, one of the cages having a standard height is placed by medium fit in accordance with a predetermined size, or the decision made during the surgery. This, in turn, makes it difficult to adjust paravertebral height. If the height of the cage is too much, two neighboring vertebrae may be spaced apart more than needed; or in the reverse situation, they may become loose in place. In the expandable systems, on the other hand, the expansion takes place over a side of the cage. The heights of the parallel surfaces of the cage contacting with the endplates change conically, but not in parallel, subsequent to expansion.

### OBJECTS OF THE INVENTION

The object of the invention is to provide an expandable cage which is moveable by means of a balloon in order to close an opening which occurs between two vertebrae, when preferred.

Another object of the present invention is to provide an expandable cage which has a unilateral movement and eliminates such incidences as dislocation and slippage between two vertebrae.

### DETAILED DESCRIPTION OF THE INVENTION

The expandable cage provided in order to achieve the object of the invention is illustrated in the accompanying figures, in which;

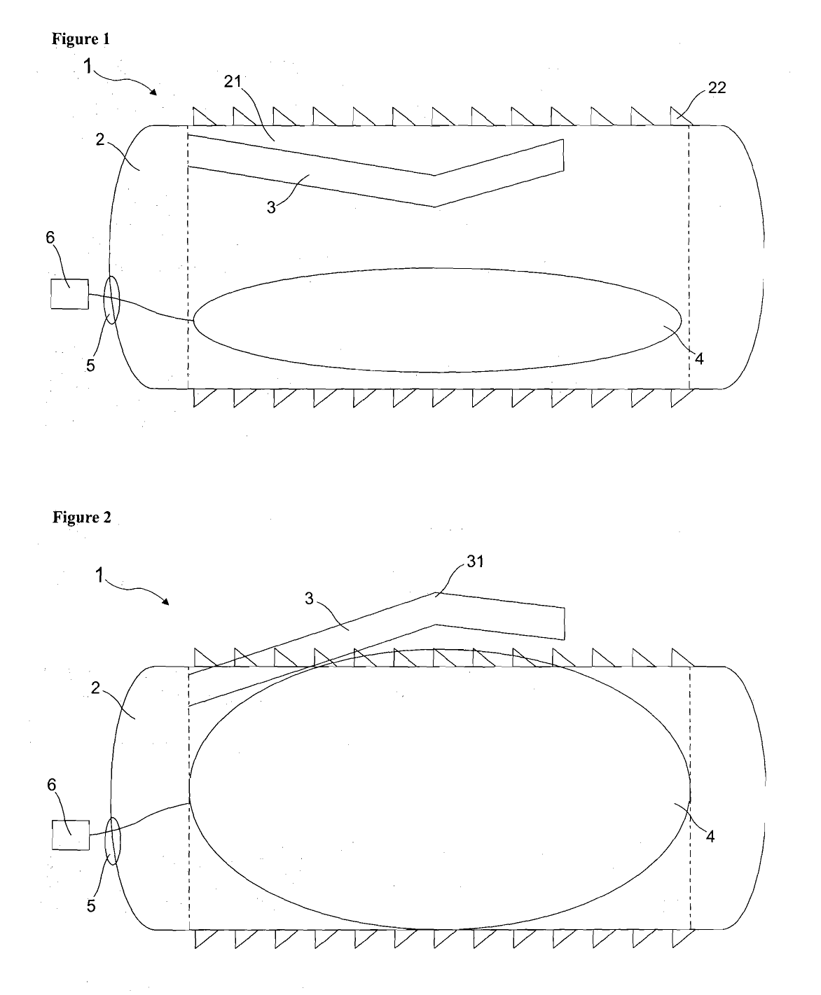

FIG. 1 is the schematic view of the expandable cage in unexpanded state.

FIG. 2 is the schematic view of the expandable cage in expanded state.

The parts in the figures are numbered individually and the references corresponding to the related part are listed below.

1. Expandable cage

2. Body
2.1. Space
2.2. Rib

3. Protrusion
3.1. Pointed tip

4. Balloon

5. Hole

6. Compression element

The expandable cage (1) which is placed between two vertebrae and expandable at a preferred ratio towards the vertebrae (i.e. in longitudinal axis) mainly comprises the following;
at least one body (2) comprising at least one space (21) and rib (22),
at least one protrusion (3) which is provided in the body (2) and which, in case of a force being applied, forms a pointed tip (3.1) out of the body (2).
at least one balloon (4) which applies the required force to the protrusion (3) such that the protrusion (3) will move unilaterally,
at least one hole (5) made in the body (2) in a way to allow directing the balloon (4) into the space (21), and
at least one compression element (6) to supply the required fluid into the balloon (4).

In one embodiment of the invention, the expandable cage (1) has a body (2). The body (2) closes, i.e. compensates, the opening between two vertebrae. The body (2) has a geometry and size such that it will be placed between two vertebrae. The body (2) is made of a biocompatible material. There exists a space (21) on the body (2). In an embodiment of the invention, the space (21) has a configuration expanding from the center, to the edges of the body (2). The space (21) may have the preferred geometry. In an embodiment of the invention, the space (21) has the same/similar geometry as/to the body (2). The depth of the space (21) is more or less the same as the height of body (2). The protrusion (3), which is to move towards the upper vertebra, is placed into the space (21). There exists ribs (22) on the surfaces of the body (2) contacting the vertebrae. Said rib (22) enables the body (2) placed between the vertebrae to firmly hold on to the vertebrae. Thanks to the rib (22), it is ensured that the body (2) placed between the vertebrae does not move between the vertebrae, or do not slip between the vertebrae.

In an embodiment of the invention, a protrusion (3) is provided in the space (21) within the body (2). The protrusion (3) is preferably configured as a hard wire, one part of which is short and the other long, and also having a wide angle between these two parts. The protrusion (3) may be as mentioned, or in the preferred geometry. The protrusion (3) is raised inside the body (2) by being moved by means of the balloon (4) in case an opening occurs between two vertebrae. The movement of the protrusion (3) is a unilateral movement. In an embodiment of the invention, the protrusion (3) moves upward from the bottom. In case of raising the protrusion (3), a pointed tip (3.1) is provided in the portion of the protrusion (3) contacting the upper vertebra. When the balloon (4) moves the protrusion (3), the pointed tip (3.1) contacts with the endplate of the upper vertebra. The portion of the protrusion (3) facing the lower vertebra is the surface where the protrusion (3) contacts with the balloon (4) in case a balloon (4) is used. The surface of the protrusion (3) contacting with the balloon (4) may have the preferred geometry. The protrusion (3) is configured such that it will not go downwards once it is moved upwards and contacted with the endplate of the vertebra by means of the balloon (4). Thus, it may be fixed where it is positioned.

An embodiment of the invention comprises a balloon (4). The balloon (4) is located under the protrusion (3) in the preferred state. Thanks to said balloon (4), the protrusion (3) can raise inside the body (2). A fluid is compressed into the balloon (4), whereby the balloon (4) is inflated. As the balloon (4) inflates, the protrusion (3) moves unilaterally inside the body (2).

In an embodiment of the invention, the body (2) is provided thereon with a hole (5). This hole (5) is made in order to allow the access of the balloon (4) to the space (21) within the body (2). In the preferred state, the balloon (4) is made to pass through the hole (5) and is inflated, and thus it enables the protrusion (3) to move inside the body (2). After this procedure is completed, the balloon (4) is withdrawn from the hole (5) and removed through the space (21). The hole (5) disposed on the body (2) and allowing access of the balloon (4) to the space (21) within the body (2) may have a preferred geometry. Said hole (5) is preferably of a geometry and size such that the balloon (4) will pass therethrough.

An embodiment of the invention comprises a compression element (6). The compression element (6) serves for compressing a fluid into the balloon (4). Thus, the balloon (4) is inflated by means of the compression element (6), thereby ensuring the movement of the protrusion (3) inside the body (2) towards the upper vertebra.

The expandable cage (1) according to the invention is used as follows. The expandable cage (1) has a body (2). The body (2) is properly fitted between two vertebrae thanks to the ribs (22) provided thereon. There exists a space (21) in the middle portion of the body (2). A plurality of protrusions (3) are arranged inside the space (21). In case an opening that cannot be compensated by the body (2) occurs between the two vertebrae, the balloon (4) is passed through the hole (5) provided on the body (2). The balloon (4) is located under the protrusion (3). Said balloon (4) is connected to the compression element (6). In cases when it is preferred that the protrusion (3) moves towards the endplate of the vertebra, a fluid is compressed into the balloon (4) by means of the compression element (6). The balloon (4) inflated by the fluid compressed by means of the compression element (6) makes the protrusion (3) move towards the endplate of the upper vertebra in the preferred height. When the movement of the protrusion (3) is stopped, the pointed tip (3.1) is anchored in the endplate, and thus the opening which occurs between two vertebrae is compensated.

1. An expandable cage which is placed between two vertebrae and expandable at a preferred ratio towards the vertebrae (i.e. in longitudinal axis) and mainly comprises:
at least one body comprising at least one space and rib,
characterized by at least one protrusion which is provided in the body and which, in case of a force being applied, forms a pointed tip out of the body, and also which is raised inside the body by being moved by means of the balloon in case an opening occurs between two vertebrae,
at least one balloon which applies the required force to the protrusion such that the protrusion will move unilaterally, and
a compression element which allows compressing a fluid into the balloon in cases when it is preferred that the protrusion moves towards the endplate of the vertebra.

2. The expandable cage according to claim 1, characterized by at least one hole made in the body in a way to allow directing the balloon into the space.

3. The expandable cage according to claim 1, characterized by at least one compression element supplying the required fluid to the balloon.

4. The expandable cage according to claim 1, characterized by a space which is provided on the body and has a configuration expanding from the center of the body towards the edges thereof.

5. The expandable cage according to claim 1, characterized by a space which has virtually the same height as the body and in which the protrusion, which is to move towards the upper vertebra, is placed.

6. The expandable cage according to claim 1, characterized by a protrusion which is arranged in the space within the body and configured as a hard wire, one part of which is short and the other long, and also having a wide angle between these two parts.

7. (canceled)

8. The expandable cage according to claim 1, characterized by a protrusion which has a unilateral movement and is capable of moving upward from the bottom inside the space.

9. The expandable cage according to claim 1, characterized by a pointed tip which is provided in the portion of the protrusion contacting the upper vertebra in case the protrusion is raised.

10. The expandable cage according to claim 1, characterized by a balloon which, when it moves the protrusion, makes the pointed tip in contact with the endplate of the upper vertebra.

11. The expandable cage according to claim 1, characterized by a protrusion which is configured such that it will not go downwards once it is moved upwards and contacted with the endplate of the vertebra by means of the balloon.

12. The expandable cage according to claim 1, characterized by a balloon which is inflated by compressing a fluid thereinto and which, as it inflates, moves the protrusion unilaterally inside the body.

13. The expandable cage according to claim 1, characterized by a hole which is made in order to allow the access of the balloon to the space within the body.

14. The expandable cage according to claim 1, characterized by a hole which is disposed on the body and allows the access of the balloon to the space within the body.

15. The expandable cage according to claim 1, characterized by a compression element which allows compressing a fluid into the balloon.

16. (canceled)

17. The expandable cage according to claim 1, characterized by a balloon which is inflated by the fluid compressed by means of the compression element makes the protrusion move towards the endplate of the upper vertebra in the preferred height.
